(19) United States
(12) Patent Application Publication
(10) Pub. No.: US 2012/0309845 A1
(43) Pub. Date: Dec. 6, 2012

(54) DOSING REGIMENS AND METHODS FOR TREATING OR PREVENTING ACUTE MYELOID LEUKEMIA

(75) Inventors: John NEUSTADT, Bozeman, MT (US); Steve PIECZENIK, Miami Beach, FL (US)

(73) Assignee: NBI Pharmaceuticals, Inc., Bozeman, MT (US)

(21) Appl. No.: 13/151,130

(22) Filed: Jun. 1, 2011

(51) Int. Cl. A61K 31/122; A61P 35/02

(52) U.S. Cl. 514/681

(57) ABSTRACT

The invention encompasses dosing regimens in which a subject is administered menatetrenone over a period of time to establish initial therapeutic baseline blood concentration of the menatetrenone followed by a maintenance therapy to maintain therapeutic blood concentrations. In other embodiments, the invention encompasses methods of treating acute myeloid leukemia in a subject in need thereof comprising administering to a subject a therapeutically effective dosing regimen of menatetrenone.

### I. FIELD OF THE INVENTION

The invention encompasses dosing regimens in which a subject is administered menatetrenone over a period of time to establish initial therapeutic baseline blood concentration of the menatetrenone followed by a maintenance therapy to maintain therapeutic blood concentrations. In other embodiments, the invention encompasses methods of treating acute myeloid leukemia in a subject in need thereof comprising administering to a subject a therapeutically effective dosing regimen of menatetrenone.

### II. BACKGROUND OF THE INVENTION

Acute myeloid leukemia (AML, also called acute myelogenous leukemia) is a malignant disease of the bone marrow in which hematopoietic precursors are arrested in an early stage of development. AML is the most common type of acute leukemia in adults. Over the past twenty years, the studies on the pathogenesis and prognosis of AML have made revolutionary progress. However, only one third of adult AML can be cured. The treatment of refractory, relapsed and elderly AML remains a major challenge. Most AML subtypes are distinguished from other related blood disorders by the presence of more than 20% blasts in the bone marrow. The underlying pathophysiology in AML consists of a maturational arrest of bone marrow cells in the earliest stages of development.

This developmental arrest results in 2 disease processes. First, the production of normal blood cells markedly decreases, which results in varying degrees of anemia, thrombocytopenia, and neutropenia. Second, the rapid proliferation of these cells, along with a reduction in their ability to undergo programmed cell death (apoptosis), results in their accumulation in the bone marrow, blood, and, frequently, the spleen and liver.

The malignant cell in AML is the myeloblast. In normal hematopoiesis, the myeloblast is an immature precursor of myeloid white blood cells; a normal myeloblast will gradually mature into a mature white blood cell. However, in AML, a single myeloblast accumulates genetic changes which “freeze” the cell in its immature state and prevent differentiation. Such a mutation alone does not cause leukemia; however, when such a “differentiation arrest” is combined with other mutations, which disrupt genes controlling proliferation, the result is the uncontrolled growth of an immature clone of cells, leading to the clinical entity of AML.

Much of the diversity and heterogeneity of AML stems from the fact that leukemic transformation can occur at a number of different steps along the differentiation pathway. Modem classification schemes for AML recognize that the characteristics and behavior of the leukemic cell (and the leukemia) may depend on the stage at which differentiation was halted.

Specific cytogenetic abnormalities can be found in many patients with AML; the types of chromosomal abnormalities often have prognostic significance. The chromosomal translocations encode abnormal fusion proteins, usually transcription factors whose altered properties may cause the “differentiation arrest.” For example, in acute myeloid leukemia, the t(15;17) translocation produces a PML-RARα fusion protein which binds to the retinoic acid receptor element in the promoters of several myeloid-specific genes and inhibits myeloid differentiation.

The clinical signs and symptoms of AML result from the fact that, as the leukemic clone of cells grows, it tends to displace or interfere with the development of normal blood cells in the bone marrow. This leads to neutropenia, anemia, and thrombocytopenia. The symptoms of AML are in turn often due to the low numbers of these normal blood elements. In rare cases, patients can develop a chloroma, or solid tumor of leukemic cells outside the bone marrow, which can cause various symptoms depending on its location.

First-line treatment of AML consists primarily of chemotherapy, and is divided into two phases: induction and postremission (or consolidation) therapy. The goal of induction therapy is to achieve a complete remission by reducing the amount of leukemic cells to an undetectable level; the goal of consolidation therapy is to eliminate any residual undetectable disease and achieve a cure. Hematopoietic stem cell transplantation is usually considered if induction chemotherapy fails or after a patient relapses, although transplantation is also sometimes used as front-line therapy for patients with high-risk disease.

All FAB subtypes except M3 are usually given induction chemotherapy with cytarabine (ara-C) and an anthracycline (such as daunorubicin or idarubicin).[32] This induction chemotherapy regimen is known as “7+3” (or “3+7”), because the cytarabine is given as a continuous IV infusion for seven consecutive days while the anthracycline is given for three consecutive days as an IV push. Up to 70% of patients will achieve a remission with this protocol. Other alternative induction regimens, including high-dose cytarabine alone or investigational agents, may also be used. Because of the toxic effects of therapy, including myelosuppression and an increased risk of infection, induction chemotherapy may not be offered to the very elderly, and the options may include less intense chemotherapy or palliative care.

The M3 subtype of AML, also known as acute acute myeloid leukemia, is almost universally treated with the drug ATRA (all-trans-retinoic acid) in addition to induction chemotherapy. Care must be taken to prevent disseminated intravascular coagulation (DIC), complicating the treatment of APL when the promyelocytes release the contents of their granules into the peripheral circulation. APL is eminently curable with well-documented treatment protocols.

The goal of the induction phase is to reach a complete remission. Complete remission does not mean that the disease has been cured; rather, it signifies that no disease can be detected with available diagnostic methods. Complete remission is obtained in about 50%-75% of newly diagnosed adults, although this may vary based on the prognostic factors described above. The length of remission depends on the prognostic features of the original leukemia. In general, all remissions will fail without additional consolidation therapy.

Even after complete remission is achieved, leukemic cells likely remain in numbers too small to be detected with current diagnostic techniques. If no further postremission or consolidation therapy is given, almost all patients will eventually relapse. Therefore, more therapy is necessary to eliminate non-detectable disease and prevent relapse—that is, to achieve a cure.

The specific type of postremission therapy is individualized based on a patient's prognostic factors (see above) and general health. For good-prognosis leukemias (i.e. inv(16), t(8;21), and t(15;17)), patients will typically undergo an additional 3-5 courses of intensive chemotherapy, known as consolidation chemotherapy. For patients at high risk of relapse (e.g. those with high-risk cytogenetics, underlying MDS, or therapy-related AML), allogeneic stem cell transplantation is usually recommended if the patient is able to tolerate a transplant and has a suitable donor. The best postremission therapy for intermediate-risk AML (normal cytogenetics or cytogenetic changes not falling into good-risk or high-risk groups) is less clear and depends on the specific situation, including the age and overall health of the patient, the patient's personal values, and whether a suitable stem cell donor is available.

For patients who are not eligible for a stem cell transplant, immunotherapy with a combination of histamine dihydrochloride (Ceplene) and interleukin-2 (Proleukin) after the completion of consolidation has been shown to reduce the absolute relapse risk by 14%, translating to a 50% increase in the likelihood of maintained remission.

For patients with relapsed AML, the only proven potentially curative therapy is a hematopoietic stem cell transplant, if one has not already been performed. In 2000, the monoclonal antibody-linked cytotoxic agent gemtuzumab ozogamicin (Mylotarg) was approved in the United States for patients aged more than 60 years with relapsed AML who are not candidates for high-dose chemotherapy.

The inventors have surprisingly found that dosing regimens of menatetrenone resulted in increased therapeutic efficacy in subjects suffering from acute myeloid leukemia.

### III. SUMMARY OF THE INVENTION

The invention generally encompasses methods of treating acute myeloid leukemia comprising administering a therapeutically effective amount of menatetrenone to a subject in need thereof.

In one embodiment, the invention encompasses a method of inducing apoptosis in leukemia cells comprising administering to a subject in need thereof a therapeutically effective amount of menatetrenone.

In another embodiment, the invention encompasses a method of inducing apoptosis in leukemia cells comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 150 mg or greater than 150 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In one embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 150 mg or greater than 150 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 125 mg or greater than 125 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 100 mg or greater than 100 mg.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 90 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 75 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 60 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 45 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 30 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 15 mg.

In another embodiment, the method further comprises administering menatetrenone in a consolidation phase after the initial induction phase but before the maintenance phase, wherein the menatetrenone is administered in a daily amount of about 250 mg.

In another embodiment, the method further comprises administering menatetrenone in a consolidation phase after the initial induction phase but before the maintenance phase, wherein the menatetrenone is administered in a daily amount of about 150 mg.

In another embodiment, the method further comprises administering menatetrenone in a consolidation phase after the initial induction phase but before the maintenance phase, wherein the menatetrenone is administered in a daily amount of about 100 mg.

In another embodiment, the method further comprises administering menatetrenone in a consolidation phase after the initial induction phase but before the maintenance phase, wherein the menatetrenone is administered in a daily amount of about 45 mg.

In another embodiment, the method further comprises administering menatetrenone in a consolidation phase after the initial induction phase but before the maintenance phase, wherein the menatetrenone is administered in a daily amount of about 30 mg.

In another embodiment, the method further comprises administering menatetrenone in a consolidation phase after the initial induction phase but before the maintenance phase, wherein the menatetrenone is administered in a daily amount of about 15 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 90 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 45 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2, years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously.

In another embodiment, the invention encompasses a method of reducing the incidence of anemia, thrombocytopenia, and neutropenia in a subject with acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 150 mg or greater than 150 mg; and

(ii) administering menatetrenone in a maintenance phase after said initial induction phase, wherein the menatetrenone is administered in a daily amount of about 15 mg to about 135 mg.

In another embodiment, the invention encompasses a method of treating acute myeloid leukemia comprising administering to a subject in need thereof a therapeutically effective amount of menatetrenone according to the following dosing regimen:

administering cytarabine in an initial induction phase for a period of at least 7 days, wherein the cytarabine is administered in a daily amount of about 100 mg/m² to about 200 mg/m²;

administering daunorubicin in an initial induction phase for a period of at least 3 days, wherein the daunorubicin is administered in a daily amount of about 10 mg/m² to about 100 mg/m²; and

administering menatetrenone in an initial induction phase for a period of at least 7 days, wherein the menatetrenone is administered in a daily amount of about 100 mg to about 250 mg.

In another embodiment, the invention encompasses administering menatetrenone in a maintenance phase after said initial induction phase, wherein the menatetrenone is administered in a daily amount of about 15 mg to about 135 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase, wherein the menatetrenone is administered in a daily amount to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously.

In another embodiment of the invention, the invention encompasses administering menatetrenone for about 180 consecutive days, optionally followed by a drug-free period of 2 to 30 consecutive days.

### IV. DETAILED DESCRIPTION OF THE INVENTION

The invention encompasses methods of treating acute myeloid leukemia comprising administering to a subject in need thereof a therapeutically effective amount of menatetrenone according to extended dose regimens. In accordance with the invention, a subject is administered an extended dose regimen of menatetrenone for a period of greater than 90 consecutive days.

An “extended dose regimen” of the invention refers to a regimen disclosed herein in which menatetrenone is administered for a period of greater than 90 consecutive days, wherein the menatetrenone is administered in at least two phases, an induction phase and a maintenance phase and optionally a consolidation phase, wherein a daily dosage of menatetrenone in a first phase is equal to or higher than a daily dosage of menatetrenone in a second phase, wherein a daily dosage of menatetrenone in a third phase is equal to or lower than the daily dosage of menatetrenone in the second phase, wherein a total daily dosage of menatetrenone in the second phase is equal to or lower than a total daily dosage of menatetrenone in the first phase, and wherein a total daily dosage of menatetrenone in the third phase is equal to or lower than the total daily dosage of menatetrenone in the second phase.

An “extended dose regimen” of the invention also refers to a regimen disclosed herein in which menatetrenone is administered for a period of greater than 90 consecutive days, wherein the menatetrenone is administered in at least two phases, wherein a total daily dosage of menatetrenone in a second phase is equal to or lower than a total daily dosage of estrogen and progestin in a first phase, and wherein a daily dosage of menatetrenone in the third phase is equal to or lower than the total daily dosage of menatetrenone in the second phase.

As used herein, “extended cycle regimen” refers to a regimen in which a menatetrenone composition is administered for a period of greater than 90 days.

As used herein, “subject” refers to any animal classified as a mammal, including humans and non-humans, such as, but not limited to, domestic and farm animals, zoo animals, sports animals, and pets.

The terms “treat” and “treatment” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a clinically significant response, without excessive levels of side effects. Treatment also includes prolonging survival as compared to expected survival if not receiving treatment.

The term “continuous” or “consecutive” in reference to “administration” means that the frequency of administration is at least once daily. Note, however, that the frequency of administration can be greater than once daily and still be “continuous,” e.g., twice or even three times daily, as long as the dosage levels as specified herein are not exceeded.

The term “daily dosage,” “daily dosage level,” “daily dosage amount,” or “daily dose” means the total amount of menatetrenone administered per day. Thus, for example, “continuous administration” of a menatetrenone to a subject at a “daily dosage level” of 90 mg means that the subject receives a total of 90 mg of menatetrenone on a daily basis, whether the menatetrenone is administered as a single 90 mg dose or, e.g., three separate 30 mg doses. A conventional means of continuously administering an menatetrenone is as a single daily oral dose at the prescribed daily dosage level.

### A. Dosages and Regimens

The invention encompasses a method of treating a subject with acute myeloid leukemia of providing an extended dosing regimen of menatetrenone, the method comprising administering to a subject in need thereof menatetrenone for a period of greater than 30 consecutive days, wherein the menatetrenone is administered in at least three phases, wherein a daily dosage of menatetrenone in a second phase is equal to or lower than a daily dosage of menatetrenone in a first phase, wherein a daily dosage of menatetrenone in a third phase is equal to or lower than the daily dosage of menatetrenone in the second phase.

The invention is also directed to a method of providing an extended dosing regimen, the method comprising administering to a subject in need thereof menatetrenone for a period of greater than 30 consecutive days, wherein the menatetrenone administered in at least two phases (e.g., induction and maintenance), wherein a total daily dosage of menatetrenone in a second phase is equal to or lower than a total daily dosage of menatetrenone in a first phase, and wherein a daily dosage of menatetrenone in the third phase is equal to or lower than the daily dosage of menatetrenone in the second phase.

In some embodiments of the invention, the daily dosage of menatetrenone in the first and second phases is equal to each other. In further embodiments of the invention, the daily dosages of menatetrenone in the first, second, and third phases are equal to each other. The daily dosage of menatetrenone can be, but is not limited to, the equivalent of 90 mg of menatetrenone daily for the first, second, and third phases.

In some embodiments of the invention, the daily dosage of menatetrenone in the second phase is lower than the daily dosage of menatetrenone in the first phase. In further embodiments of the invention, the daily dosage of menatetrenone in the third phase is lower than the daily dosage of menatetrenone in the second phase.

In some embodiments of the invention, the daily dosage of menatetrenone in the second phase is equal to the daily dosage of menatetrenone in the first phase and the daily dosage of menatetrenone in the third phase is lower than the daily dosage of menatetrenone in the second phase. In other embodiments of the invention, the daily dosage of menatetrenone in the second phase is lower than the daily dosage of menatetrenone in the first phase and the daily dosage of menatetrenone in the third phase is lower than the daily dosage of menatetrenone in the second phase.

The daily dosage of menatetrenone in the first phase can be, but is not limited to, about 45 mg to about 1000 mg, about 60 mg to about 500 mg, or about 90 mg to about 150 mg of menatetrenone. In certain embodiment, the daily dosage in any phase is about 15 mg, 30 mg, 45 mg, 60 mg, 75 mg, 90 mg, 105 mg, 120 mg, 135 mg, 150 mg, 225 mg, 450 mg, 600 mg, 800 mg, or 1000 mg. For example, the daily dosage of menatetrenone in the first initial phase can be 90 mg to 150 mg. The daily dosage of menatetrenone in the second phase can be, but is not limited to, about 30 mg to about 250 mg, about 60 mg to about 150 mg, or about 90 mg to about 135 mg of menatetrenone. For example, the daily dosage of menatetrenone in the second phase can be about 60 mg. The daily dosage of menatetrenone in the third phase can be, but is not limited to, about 15 mg to 125 mg, about 30 mg to about 90 mg, or about 45 mg to about 60 mg. For example, the daily dosage of menatetrenone in the third phase can be the equivalent of 45 mg.

In some embodiments of the invention, the method of providing an extended cycle regimen further includes a drug-free period. The drug-free period can be, but is not limited to, 2 to 10 consecutive days. The drug-free period can be 2 to 8 consecutive days. For example, the drug-free period can be 3, 5 or 7 days. The drug-free period can be a non-administration or administration of a placebo. The drug-free period can include administration of other active ingredients.

Examples of other additional pharmaceutically active ingredients or agents include, but are not limited to, vitamin D or vitamin D analogues; one or more of the B complex vitamins, such as vitamin B3 (niacin (i.e., nicotinic acid and/or nicotinamide)), vitamin B9 (folic acid or folate), vitamin B6 and/or vitamin B12; minerals such as, for example, calcium; iron (e.g., ferrous iron, such as, e.g., ferrous sulfate, ferrous fumarate, ferrous gluconate, or an iron glycine amino acid chelate),

These additional active agents can be administered during the period of administration of menatetrenone or the drug-free period. For example, vitamin D and/or calcium can be administered during the drug-free period. The active ingredients can be provided in the same, different, or separate dosage forms.

In some embodiments of the invention, the administration of an extended dose regimen of the invention is followed by monophasic administration of an menatetrenone. As used herein, “monophasic” refers to the continuous use of one particular dose of menatetrenone during the period of administration of the dosage form of the menatetrenone. In some aspects of the invention, the administration of an extended dose regimen is followed by monophasic administration of an menatetrenone continuously. In some aspects of the invention, the administration of an extended dose regimen is followed by monophasic administration of an menatetrenone for a period of greater than 30 or 31 consecutive days.

In some embodiments of the invention, the administration of an extended dose regimen is followed by monophasic administration of an menatetrenone for a period of about 350 to about 370 consecutive days, of about 260 to about 280 consecutive days, of about 175 to about 190 consecutive days, or of about 60 to about 110 consecutive days. The monophasic administration of menatetrenone can be optionally followed either by a drug-free period of, e.g., 2 to 10 consecutive days or by administration of menatetrenone for a period of, e.g., 2 to 10 consecutive days.

### B. Methods of Treatment

The invention generally encompasses methods of treating acute myeloid leukemia comprising administering a therapeutically effective amount of menatetrenone to a subject in need thereof.

In one embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 150 mg or greater than 150 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 125 mg or greater than 125 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 100 mg or greater than 100 mg.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 90 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 75 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 60 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 45 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 30 mg.

In another embodiment, the invention encompasses a method of treating acute myeloid leukemia comprising following the initial induction phase administering to the subject a therapeutically effective amount of menatetrenone according to the following dosing regimen: (ii) administering menatetrenone in a maintenance phase after said initial induction phase, wherein the menatetrenone is administered in a daily amount of about 15 mg.

In another embodiment, the method further comprises administering menatetrenone in a consolidation phase after the initial induction phase but before the maintenance phase, wherein the menatetrenone is administered in a daily amount of about 250 mg.

In another embodiment, the method further comprises administering menatetrenone in a consolidation phase after the initial induction phase but before the maintenance phase, wherein the menatetrenone is administered in a daily amount of about 150 mg.

In another embodiment, the method further comprises administering menatetrenone in a consolidation phase after the initial induction phase but before the maintenance phase, wherein the menatetrenone is administered in a daily amount of about 100 mg.

In another embodiment, the method further comprises administering menatetrenone in a consolidation phase after the initial induction phase but before the maintenance phase, wherein the menatetrenone is administered in a daily amount of about 45 mg.

In another embodiment, the method further comprises administering menatetrenone in a consolidation phase after the initial induction phase but before the maintenance phase, wherein the menatetrenone is administered in a daily amount of about 30 mg.

In another embodiment, the method further comprises administering menatetrenone in a consolidation phase after the initial induction phase but before the maintenance phase, wherein the menatetrenone is administered in a daily amount of about 15 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 90 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 8 weeks, wherein the menatetrenone is administered in a daily amount of about 45 mg to about 500 mg; and

(ii) administering menatetrenone in a maintenance phase after said initial induction phase, wherein the menatetrenone is administered in a daily amount of about 15 mg to about 135 mg.

In another embodiment, the method further comprises administering menatetrenone in a consolidation phase after the initial induction phase but before the maintenance phase, wherein the menatetrenone is administered in a daily amount of about 45 mg to about 250 mg.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2, years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase, wherein the menatetrenone is administered in a daily amount to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In another embodiment, the invention encompasses a method of treating acute myeloid leukemia comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and

(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

In another embodiment, the method further comprises administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

In certain embodiments, the amount of menatetrenone administered in the maintenance phase is less than the amount of menatetrenone administered in the initial induction phase.

In certain embodiments, the initial induction phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months or 1 year.

In certain embodiments, the initial induction phase period is about 30 to 120 days.

In certain embodiments, the initial induction phase period is about 60 to 90 days.

In certain embodiments, the maintenance phase is 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 4 months, 5 months, 6 months, 8 months, 10 months, 1 year, 2 years, 3 years, or 5 years.

In certain embodiments, the maintenance phase period is at least 60 days.

In certain embodiments, the maintenance phase period is at least 90 days.

In certain embodiments, the maintenance phase period is at least 120 days.

In certain embodiments, the consolidation phase period is at least 60 days.

In certain embodiments, the consolidation phase period is at least 120 days.

In certain embodiments, the consolidation phase period is at least 180 days.

In certain embodiments, the menatetrenone is administered intravenously, orally, or subcutaneously

In another embodiment of the invention, the invention encompasses administering menatetrenone for about 180 consecutive days, optionally followed by a drug-free period of 2 to 30 consecutive days.

In certain embodiments, the menatetrenone is administered according to the following dosing regimen:

(i) administering menatetrenone in an initial induction phase for a period of at least 7 days, wherein the menatetrenone is administered in a daily amount of about 100 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of inducing apoptosis and differentiation in leukemic cells comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of treating acute myeloid leukemia in a subject refractive to traditional chemotherapy (e.g., a subject not able to tolerate chemotherapeutic drugs, for example, lenalidomide or azacitidine) comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of treating acute myeloid leukemia in a subject over 65 years old who cannot tolerate intensive myeloablative chemotherapy and/or stem cell transplantation comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

In other embodiments, the invention encompasses a method of improving hematopoeisis comprising administering to a subject in need thereof a therapeutically effective amount of menatetrenone according to the following dosing regimen:

(i) administering menatetrenone in an initial induction phase for a period of at least 7 days, wherein the menatetrenone is administered in a daily amount of about 60 mg to about 500 mg; and

(ii) administering menatetrenone in a maintenance phase after said initial induction phase, wherein the menatetrenone is administered in a daily amount of about 15 mg to about 135 mg.

In certain embodiments, the method further comprises administering menatetrenone in a consolidation phase after the initial induction phase but before the maintenance phase, wherein the menatetrenone is administered in a daily amount of about 45 mg to about 250 mg.

### C. Modes of Administration and Compositions

The menatetrenone is administered in the conventional manner by any route where they it is active. For example, administration can be by, but is not limited to, oral, parenteral, subcutaneous, intravenous, intramuscular, intraperitoneal, transdermal, buccal, or ocular routes, or by inhalation, by depot injections, or by implants. Thus, the dosage forms for the menatetrenone can be, but are not limited to, sublingual, injectable (including short-acting, depot, implant and pellet forms injected subcutaneously or intramuscularly), rectal suppositories, intrauterine devices, and transdermal forms such as patches and creams.

Thus, pharmaceutical compositions containing menatetrenone and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder. It is known in the art that the active ingredients can be contained in such compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, “Modern Pharmaceutics”, Banker & Rhodes, Marcel Dekker, Inc. 1979; and “Goodman & Gilman's The Pharmaceutical Basis of Therapeutics,” 6.sup.th Edition, MacMillan Publishing Co., New York 1980 can be consulted.

For oral administration, the menatetrenone can be formulated by combining with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

The pharmaceutical compositions of menatetrenone also can comprise suitable solid or gel phase carriers or excipients such as calcium carbonate, calcium phosphate, various sugars, starches, cellulose derivatives, gelatin, and polymers such as, e.g., polyethylene glycols.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All compositions for oral administration should be in dosages suitable for such administration.

For buccal administration, the menatetrenone compositions can take the form of tablets or lozenges formulated in conventional manner.

For administration by inhalation, the menatetrenone for use according to the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The menatetrenone can be formulated for parenteral administration by injection, e.g., by bolus injection or continuous infusion. The menatetrenone can be administered by continuous infusion subcutaneously over a period of about 15 minutes to about 24 hours. Compositions for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The menatetrenone can also be formulated in rectal compositions such as suppositories or retention enemas, e.g., containing conventional suppository bases such as cocoa butter or other glycerides.

In addition to the compositions described previously, the menatetrenone can also be formulated as a depot preparation. Such long acting compositions can be administered by implantation (e.g., subcutaneously or intramuscularly) or by intramuscular injection. Depot injections can be administered at about 1 to about 6 months or longer intervals. Thus, for example, the menatetrenone can be formulated with suitable polymeric or hydrophobic materials (e.g., as an emulsion in an acceptable oil) or ion exchange resins, or as sparingly soluble derivatives, for example, as a sparingly soluble salt.

For transdermal administration, the menatetrenone can be applied by any transdermal, therapeutic system that is consequently supplied to the organism, such as, for example, as a transdermal patch, transdermal cream or plaster. For example, the menatetrenone can be formulated as a transdermal patch. The preparation and use of transdermal patches are well known to those of skill in the art and are available in different designs, including matrix-type or reservoir-type designs. In addition to the estrogen and/or progestin, transdermal patches can contain additional components such as penetration-enhancing agents and/or additional excipients that are conventionally employed, such as, e.g., carriers, gelling agents, suspending agents, dispersing agents, preservatives, stabilizers, wetting agents, emulsifying agents, and the like.

The menatetrenone can also be administered with other active ingredients. The drug-free period or the unopposed estrogen interval can also include administration of other active ingredients. For example, as described above, menatetrenone can also be administered with vitamin D and/or calcium in the extended cycle regimens. Alternatively, vitamin D and/or calcium can be administered in the extended cycle regimens during the unopposed menatetrenone interval following administration of menatetrenone. The form of vitamin D and of calcium used in the invention would be well known to those of skill in the art, as would the amount. For example, calcium can be administered in the form of calcium carbonate, at a dosage level of e.g., 500 mg.

In some embodiments of the invention, the menatetrenone is in a oral, transdermal, or injectable liquid dosage form. For example, the menatetrenone can be in an oral dosage form or a transdermal dosage form.

In some embodiments of the invention, each phase of the regimen of the invention can be administered in a separate, single dosage form. In other aspects of the invention, each phase of the regimen of the invention can be administered in one or more separate dosage forms. For example, each phase can be administered using a transdermal device (such as a patch).

### D. Kits

The dosages or compositions for the extended cycle regimens of the invention can be provided in the form of a kit or package, with the dosages arranged for proper sequential administration. For example, in the oral form of the composition, the invention provides a pharmaceutical package, which contains multiple dosage units in a synchronized, fixed sequence, wherein the sequence or arrangement of the dosage units corresponds to the stages of daily administration.

Thus, for example, the pharmaceutical compositions useful in the invention can be provided in kit form containing greater than 30 or 31 tablets intended for ingestion on successive days. In some embodiments, the kit further contains, e.g., 2 to 10 tablets, intended for ingestion on successive days following the ingestion of the greater than 30 or 31 tablets. Administration is daily for a period of greater than 30 or 31 consecutive days using tablets containing menatetrenone, and, in some embodiments, is followed by administration that is daily for, e.g., 2 to 10 consecutive days using either placebo tablets or tablets containing no menatetrenone. For example, administration can be for 40-190 consecutive days, using tablets containing menatetrenone, followed by administration for, e.g., at least 2-10 days using tablets containing no menatetrenone. As another example, administration can be for 75-95 days, using tablets containing menatetrenone, followed by administration for, e.g., at least 2-10 days using tablets containing no menatetrenone. As yet another example, administration can be for 168-186 days, using tablets containing menatetrenone, followed by administration for, e.g., at least 2-10 days using tablets containing no menatetrenone.

Some embodiments of the invention provide a pharmaceutical kit comprising a first transdermal device capable of providing a daily dosage of menatetrenone; a second transdermal device capable of providing a daily dosage of menatetrenone that is equal to or higher than that of the first transdermal device and capable of providing a total daily dosage of menatetrenone that is higher than that of the first transdermal device; and a third transdermal device capable of providing a daily dosage of menatetrenone that is equal to or higher than that of the second transdermal device and capable of providing a total daily dosage of estrogen and progestin that is higher than that of the second transdermal device; wherein the transdermal devices are capable of providing menatetrenone for a period of greater than 30 or 31 consecutive days.

Some embodiments of the invention provide a pharmaceutical kit comprising a first oral dosage capable of providing a daily dosage of menatetrenone; a second oral dosage capable of providing a daily dosage of menatetrenone that is equal to or lower than that of the first oral dosage and capable of providing a total daily dosage of menatetrenone that is higher than that of the first oral dosage; and a third oral dosage capable of providing a daily dosage of menatetrenone that is equal to or lower than that of the second oral dosage and capable of providing a total daily dosage of menatetrenone that is higher than that of the second oral dosage; wherein the oral dosages are capable of providing menatetrenone for a period of greater than 30 or 31 consecutive days.

The pharmaceutical kits of the invention can further include instructions for proper sequential administration in accordance with the regimens of the invention.

The invention also encompasses a method of delivering a pharmaceutical composition for an extended dose regimen of the invention to a patient in need thereof, the method comprising (a) registering in a computer readable medium the identity of a physician permitted to prescribe a pharmaceutical composition for an ascending-dose extended cycle regimen; (b) providing the patient with counseling information concerning the risks attendant to the pharmaceutical composition; (c) obtaining informed consent from the patient to receive the pharmaceutical composition despite the attendant risks; (d) registering the patient in a computer readable medium after obtaining their informed consent; and (e) permitting the patient access to the pharmaceutical composition.

The drug delivery methods of the invention involve, inter alia, registering in a computer readable storage medium physicians who are qualified to prescribe the ascending-dose extended cycle regimen of the present invention. Once registered in the computer readable storage medium, the physician can be eligible to prescribe the pharmaceutical composition to a patient in need thereof. Generally speaking, in order to become registered in the computer readable storage medium, the physician may be required to comply with various aspects of, for example, providing patient education and counseling. The registration of the physician in the computer readable storage medium can be achieved by providing the physician, for example, by mail, facsimile transmission, or on-line transmission, with a registration card or form, preferably together with educational materials concerning the pharmaceutical composition of the present invention. The physician can complete the registration card or form by providing information requested therein, and the registration card or form can be returned to the manufacturer or distributor of the pharmaceutical composition of the present invention, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, the physician may determine that the patient's condition can be improved by the administration of the pharmaceutical composition of the invention. Prior to prescribing the pharmaceutical composition of the invention, the physician can counsel the patient, for example, on the various risks and benefits associated with the pharmaceutical composition of the invention. The patient can be provided full disclosure of all the known and suspected risks associated with the pharmaceutical composition of the present invention. Such counseling can be provided verbally, as well as in written form. In some embodiments, the physician can provide the patient with literature materials on the pharmaceutical composition of the present invention, such as product information, educational materials, and the like.

In addition to receiving counseling on the risks attendant to the pharmaceutical composition of the present invention, the methods of the invention further require the patient to fill out an informed consent form which is signed by the patient. Upon the completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the pharmaceutical composition of the present invention. Thus, the computer readable storage medium can serve to deny access to patients who fail to abide by the methods of the present invention. In some embodiments, access to the pharmaceutical composition of the present invention is in the form of a prescription, wherein the prescribing physician is registered in a computer readable storage medium, has provided counseling to the patient concerning the attendant risks of the pharmaceutical composition of the present invention, and has obtained informed consent from the patient, prior to prescribing the pharmaceutical composition of the present invention to the patient in need thereof.

All of the various aspects, embodiments and options described herein can be combined in any and all variations. The following examples are illustrative, but not limiting, of the method and compositions of the present invention. Other suitable modifications and adaptations of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance with the following claims and their equivalents.

### V. EXAMPLES

### Prophetic Example 1

Patients with MDS and post-MDS AML are eligible for evaluation. Menatetrenone is administered in a range from 90 to 250 mg/day orally for 30 days followed by 30 to 150 mg/day orally for 180 days. Menatetrenone administration shows improvement of cytopenia, an increased number of peripheral neutrophils, and improvement of pancytopenia and an increased number of neutrophils.

A case of treating RAEB-T with menatetrenone shows a reduction of the peripheral blast cell number by an oral treatment of 90 mg/day for 3 months.

In a case of a patient with post-MDS AML with chronic renal failure, who required hemodialysis and periodic erythropoietin injections, an oral dose of menatetrenone is administered in a range from 45 to 250 mg/day orally for 30 days followed by 90 to 150 mg/day orally for 180 days, leads to transfusion independence.

### Prophetic Example 2

Freshly isolated leukemia cells from patients with AML will first be treated with MK4 at 135 mg for 48 hours, and their morphological changes investigated. Because the number of leukemia cells that could be isolated from a patient is limited, investigators will use flow cytometry to establish a convenient one-step method for evaluating the cytocidal effect of MK4 and assessing the cytogram pattern. In addition to flow cytometry, the researchers will evaluate changes in DNA fragments using Fluorescein, an ApopTag kit from Oncor in Gaithersburg, Md.

In the AML subject, the leukemic cells should be completely eliminated after exposure to MK4. Additionally, the ApopTag method should reveal treatment of cells with 135 mg/day MK4 induces significant enhancement of apoptosis within 48 hours compared to untreated controls. In addition to its cytocidal effect on freshly isolated leukemia cells, MK4 shows cytocidal and apoptosis-inducing effects against NB4 cells in a concentration-dependent manner. For additional studies the researchers will use flow cytograms exclusively to monitor the cytocidal effect of MK4 on leukemia cells. MK4 should selectively eliminate the leukemia cell population.

This study will demonstrate that MK4 has a potent apopotosis-inducing effect on freshly isolated leukemia cells. The MK4 dose achieved in this study (135 mg/day initially for an induction period) will then be tested in vivo, since serum concentration of MK4 should be maintained after oral administration of 135 mg of MK4.

### Prophetic Example 3

This study will evaluate general categories of AML treatment phases: Induction, Consolidation, and Maintenance therapies. The treatment of patients with AML includes at least one course of intensive myelosuppressive induction chemotherapy.

Cytarabine (AraC) is the cornerstone of induction therapy and consolidation therapy for AML. A standard form of induction therapy consists of AraC (100-200 mg/m²), administered by a continuous infusion for 7 days, combined with daunorubicin, administered intravenously for 3 days (the 3+7 induction regimen). This therapy has been reported to induce a complete remission (CR) in 65% to 75% of patients aged 18 to 60 years. This approach results in a long-term disease-free survival of ˜30%, with a treatment-related mortality (i.e., the percentage of patients who died during induction) of 5% to 10%.

This study will test a new dosing regimen AraC (100-200 mg/m²), administered by a continuous infusion for 7 days, combined with daunorubicin (20-50 mg/m²), administered intravenously for 3 days (the 3+7 induction regimen) combined with MK4 (135 mg/day peroral) administered for at least 7 days.

Consolidation therapy comprises treatment with additional courses of intensive chemotherapy after the patient has achieved a complete remission, usually with higher doses of the same drugs as were used during the induction period. The median disease-free survival for patients who received only the induction therapy is 4 to 8 months. However, 35% to 50% of adults aged <60 years who receive consolidation treatment survive 2 to 3 years.

High-dose AraC (2-3 g/m2) is now a standard consolidation therapy for patients aged <60 years. Despite substantial progress in the treatment of newly diagnosed AML, 20% to 40% of patients do not achieve remission with the standard induction chemotherapy, and 50% to 70% of first complete remission patients are expected to relapse within 3 years. The optimum strategy at the time of relapse, or for patients with the resistant disease, remains uncertain. Allogeneic stem cell transplantation has been established as the most effective form of antileukemic therapy in patients with AML in first or subsequent remission. The augmentation of the cytogenetic risk stratification by molecular prognostic markers may help define additional subgroups of AML patients who will benefit from the intensified chemotherapy. This study will test a new consolidation dosing regimen AraC (2-3 g/m²), administered by a continuous infusion combined with MK4 (90 mg/day peroral) administered for at least 8 weeks.

Maintenance therapy, which is considered less myelosuppressive than the induction and consolidation forms of treatment, is used in patients who have previously obtained complete remission. It is a strategy to further reduce the number of residual leukemic cells and prevent a relapse. However, its role in the routine management of AML patients is controversial and depends mainly on the intensity of the induction and consolidation therapies. Maintenance therapy will entail administration of MK4 (45 mg/day orally) for at least 6 months.

Application of the compounds, compositions and methods of the present invention for the medical or pharmaceutical uses described can be accomplished by any clinical, medical, and pharmaceutical methods and techniques as are presently or prospectively known to those skilled in the art. It will therefore be appreciated that the various embodiments which have been described above are intended to illustrate the invention and various changes and modifications can be made in the invention method without departing from the spirit or scope thereof.

Having now fully described this invention, it will be understood to those of ordinary skill in the art that the same can be performed within a wide and equivalent range of conditions, compositions, and other parameters without affecting the scope of the invention or any embodiments thereof. All patents, patent applications, and publications cited herein are frilly incorporated by reference herein in their entirety.

What is claimed is:

1. A method of treating acute myeloid leukemia comprising administering to a subject in need thereof a therapeutically effective amount of menatetrenone according to the following dosing regimen:
(i) administering menatetrenone in an initial induction phase for a period of at least eight weeks, wherein the menatetrenone is administered in a daily amount of about 60 mg to about 500 mg; and
(ii) administering menatetrenone in a maintenance phase after said initial induction phase, wherein the menatetrenone is administered in a daily amount of about 15 mg to about 135 mg.

2. The method of claim 1, further comprising administering menatetrenone in a consolidation phase after the initial induction phase but before the maintenance phase, wherein the menatetrenone is administered in a daily amount of about 45 mg to about 250 mg.

3. The method of claim 1, wherein the amount of menatetrenone administered in the maintenance phase is less than the amount of menatetrenone administered in the initial induction phase.

4. The method of claim 1, wherein the initial induction phase period is about 30 to 120 days.

5. The method of claim 1, wherein the initial induction phase period is about 60 to 90 days.

6. The method of claim 1, wherein the maintenance phase period is at least 60 days.

7. The method of claim 1, wherein the maintenance phase period is at least 90 days.

8. The method of claim 1, wherein the maintenance phase period is at least 120 days.

9. The method of claim 2, wherein the consolidation phase period is at least 60 days.

10. The method of claim 2, wherein the consolidation phase period is at least 120 days.

11. The method of claim 2, wherein the consolidation phase period is at least 180 days.

12. The method of claim 1, wherein the menatetrenone is administered intravenously, orally, or subcutaneously.

13. A method of treating acute myeloid leukemia comprising administering to a subject in need thereof a therapeutically effective amount of menatetrenone according to the following dosing regimen:
(i) administering menatetrenone in an initial induction phase for a period of at least 7 days, wherein the menatetrenone is administered in a daily amount to achieve a peak plasma concentration (Cmax) of about 60 ng/mL to about 760 ng/mL; and
(ii) administering menatetrenone in a maintenance phase after said initial induction phase, wherein the menatetrenone is administered in a daily amount to achieve a peak plasma concentration (Cmax) of about 30 ng/mL to about 380 ng/mL.

14. The method of claim 13, further comprising administering menatetrenone in a consolidation phase after the initial induction phase but before the maintenance phase, wherein the menatetrenone is administered in a daily amount to achieve a peak plasma concentration (Cmax) of about 30 ng/mL to about 380 ng/mL.

15. The method of claim 13, wherein the amount of menatetrenone administered in the maintenance phase is less than the amount of menatetrenone administered in the initial induction phase.

16. The method of claim 13, wherein the initial induction phase period is about 30 to 120 days.

17. The method of claim 13, wherein the initial induction phase period is about 60 to 90 days.

18. The method of claim 13, wherein the maintenance phase period is at least 60 days.

19. The method of claim 13, wherein the maintenance phase period is at least 90 days.

20. The method of claim 13, wherein the maintenance phase period is at least 120 days.

21. The method of claim 14, wherein the consolidation phase period is at least 60 days.

22. The method of claim 14, wherein the consolidation phase period is at least 120 days.

23. The method of claim 14, wherein the consolidation phase period is at least 180 days.

24. A method of treating acute myeloid leukemia comprising administering to a subject in need thereof a therapeutically effective amount of menatetrenone according to the following dosing regimen:
(i) administering menatetrenone in an initial induction phase for a period of at least 7 days, wherein the menatetrenone is administered in a daily amount of about 60 mg to about 500 mg to achieve a peak plasma concentration (Cmax) of about 60 ng/mL to about 760 ng/mL; and
(ii) administering menatetrenone in a maintenance phase after said initial induction phase, wherein the menatetrenone is administered in a daily amount of about 15 mg to about 135 mg to achieve a peak plasma concentration (Cmax) of about 30 ng/mL to about 380 ng/mL.

25. The method of claim 24, further comprising administering menatetrenone in a consolidation phase after the initial induction phase but before the maintenance phase, wherein the menatetrenone is administered in a daily amount of about 45 mg to about 250 mg to achieve a peak plasma concentration (Cmax) of about 30 ng/mL to about 380 ng/mL.

26. The method of claim 24, wherein the amount of menatetrenone administered in the maintenance phase is less than the amount of menatetrenone administered in the initial induction phase.

27. The method of claim 24, wherein the initial induction phase period is about 30 to 120 days.

28. The method of claim 24, wherein the initial induction phase period is about 60 to 90 days.

29. The method of claim 24, wherein the maintenance phase period is at least 60 days.

30. The method of claim 24, wherein the maintenance phase period is at least 90 days.

31. The method of claim 24, wherein the maintenance phase period is at least 120 days.

32. The method of claim 25, wherein the consolidation phase period is at least 60 days.

33. The method of claim 25, wherein the consolidation phase period is at least 120 days.

34. The method of claim 25, wherein the consolidation phase period is at least 180 days.

35. The method of claim 24, wherein the menatetrenone is administered intravenously, orally, or subcutaneously.
